(19) United States
(12) Patent Application Publication
(10) Pub. No.: US 2008/0125442 A1
(43) Pub. Date: May 29, 2008

(54) Cathepsin K Inhibitors and Atherosclerosis

(76) Inventor: Michael David Percival, Montreal West (CA)

Correspondence Address: MERCK AND CO., INC, P O BOX 2000, RAHWAY, NJ, 07065-0907, US

(21) Appl. No.: 11/795,444

(22) PCT Filed: Jan. 17, 2006
(86) PCT No.: PCT/CA06/00055

§ 371 Date: Jul. 17, 2007

Related U.S. Application Data
(60) Provisional application No. 60/644,938, filed on Jan. 19, 2005.

(51) Int. Cl. A61K 31/495; A61K 31/164; C07D 241/04; C07D 211/00; A61P 9/00; A61P 9/10; A61K 31/451; C07C 243/00

(52) U.S. Cl. 514/255.03; 514/614; 564/147; 544/358; 514/331; 546/192

(57) ABSTRACT

This invention relates to a genus of compounds, represented by the formula (I) diagrammed below, wherein the meanings of R¹, R², R³, R⁴, R⁵, R⁶, R⁷, R⁸, D and n are indicated therein, which are inhibitors of cathepsin K. These compounds are useful for treating or preventing atherosclerosis and atherosclerotic cardiovascular disease.

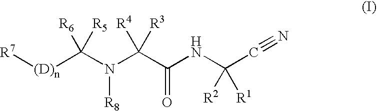

### BACKGROUND OF THE INVENTION

This invention relates to the treatment of atherosclerosis by the administration of a cathepsin K inhibitor, either as a single agent or in combination with other agents.

Atherosclerosis is a common disorder of the arteries. Fat, cholesterol, and other substances accumulate in the walls of arteries and form “atheromas” or plaques. Eventually, this fatty tissue can erode the wall of the artery, diminish its elasticity and interfere with blood flow. Plaques can also rupture, causing debris to migrate downstream within an artery. This is a common cause of heart attack and stroke. Clots can also form around the plaque deposits, further interfering with blood flow and posing added danger if they break off and travel to the heart, lungs, or brain.

It has been suggested that cathepsin K plays a role in the vessel wall remodeling that occurs during atherosclerosis and that genetic ablation of cathepsin K reduces the progression of atherosclerosis in a mouse model. Normal arteries contain little or no cathepsin K, but atheroma macrophages and smooth muscle cells contain abundant cathepsin K, see Sukhova G K, Shi G P, Simon D I, Chapman H A, Libby P. Expression of the elastolytic cathepsins S and K in human atheroma and regulation of their production in smooth muscle cells. J Clin Invest. 1998, 102:576-83. The elastolytic activity of atheromatous tissue is increased approximately two-fold compared to normal arteries, mostly due to cysteine proteases. Furthermore, cathepsin K is among the genes differentially expressed in human coronary artery disease, see Archacki S R, Angheloiu G, Tian X L, Tan F L, DiPaola N, Shen G Q, Moravec C, Ellis S, Topol E J, Wang Q. Identification of new genes differentially expressed in coronary artery disease by expression profiling. Physiol Genomics. 2003, 15: 65-74. After crossing the ApoE-deficient mouse with the cathepsin K null mouse and 26 weeks on a normal diet, the total plaque area per aortic arch was reduced 1.6-fold and the plaques showed a delayed progression in the double cathepsin K/ApoE null animals versus ApoE deficient animals, see Cleutjens K B, Lutgens E, Sijbers A M, Faber B C, Black D, Long C J; Fisher A, Saftig P, Daemen M J. Targeted disruption of the cathepsin K gene results in reduced atherosclerotic plaque progression. Circulation 106 (19 Supplement): pII-121 Nov. 5, 2002, see Cleutjens K B, Lutgens E, Faber B C, Heeneman S, Gijbels M, Sijbers A M, Fisher A, Long C J; Saftig P, Daemen M J. Disruption of the cathepsin K gene reduces the progression of atherosclerosis. Thrombosis and Vascular Biology v. 23, #5, pg a-66, Poster P377, 2003. The fibrous plaque in the cathepsin K double null mice was 4-fold thicker than ApoE deficient mice and the collagen content was increased 2-fold, suggesting a reduced vulnerability to plaque rupture. These data suggest that cathepsin K inhibitors may be useful treatments for atherosclerosis and related disorders.

The present data shows that cathepsin K null mice have significantly lower plasma total cholesterol, triglycerides and leptin levels following feeding 12 weeks of a high fat, high carbohydrate diet. Cholesterols, triglycerides and leptin are all considered risk factors for the development of atherosclerosis. See Fruchart J C, Nierman M C, Stroes E S, Kastelein J J, Duriez P, New risk factors for atherosclerosis and patient risk assessment. Circulation. 2004; 109(23 Suppl 1):III-15-9.

Despite significant therapeutic advances in the treatment and prevention of atherosclerosis and ensuing atherosclerotic disease events, such as the improvements that have been achieved with HMG-CoA reductase inhibitors, further treatment options are clearly needed. The instant invention addresses that need by providing compounds, compositions and methods for the treatment or prevention of atherosclerosis as well as related conditions.

### SUMMARY OF THE INVENTION

This invention relates to the treatment of atherosclerosis by the administration of a cathepsin K inhibitor, either as a single agent or in combination with other agents.

### DETAILED DESCRIPTION OF THE INVENTION

The instant invention relates to the treatment of atherosclerosis by the administration of a cathepsin K inhibitor, either as a single agent or in combination with other agents. In an embodiment of the invention, the cathepsin K inhibitor is a compound of formula I:

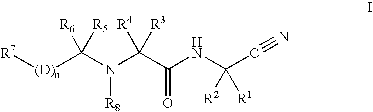

and the pharmaceutically acceptable salts, esters and solvates thereof wherein:
wherein R¹ is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
R² is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
or R¹ and R² can be taken together with the carbon atom to which they are attached to form a C₃₋₈ cycloalkyl or heterocyclyl ring wherein said ring system is optionally substituted with one or two substituents independently selected from C₁₋₆ alkyl, hydroxyalkyl, haloalkyl, or halo;

### R³ is hydrogen, C₁₋₆ alkyl or C₂₋₆ alkenyl wherein said alkyl and alkenyl groups are optionally substituted with C₃₋₆ cycloalkyl or one to six halo;

### R⁴ is hydrogen, C₁₋₆ alkyl or C₂₋₆ alkenyl wherein said alkyl and alkenyl groups are optionally substituted with C₃₋₆ cycloalkyl or one to six halo;

or R³ and R⁴ can be taken together with the carbon atom to which they are attached to form a C₃₋₈ cycloalkyl ring, C₃₋₈ cycloalkenyl ring, or five to seven membered heterocyclyl wherein said cycloalkyl, cycloalkenyl and heterocyclyl groups are optionally substituted with one or two substitutents independently selected from C₁₋₆ alkyl, halo, hydroxyalkyl, hydroxy, alkoxy or keto;

### R⁵ is selected from hydrogen or C₁₋₆ alkyl substituted with 1-6 halo;

R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with one, two, or three substituents independently selected from halo, C₁₋₆ alkyl, C₁₋₆ haloalkyl, C₃₋₆ cycloalkyl, haloalkoxy, —SR⁹, —SR¹², —SOR⁹, —SOR¹², —SO₂R⁹, —SO₂R¹², —SO₂CH(R¹²)(R¹¹), —OR¹², —N(R¹⁰)(R¹¹), cyano, or aryl which is optionally substituted with —SO₂R¹²;
each D is independently C₁₋₃ alkyl, C₂₋₃ alkenyl, C₂₋₃ alkynyl, aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from C₁₋₆ alkyl, haloalkyl, halo, keto, alkoxy, —SR⁹, —SR¹², —OR⁹, —OR¹², N(R¹²)₂, —SO₂R⁹, or —SO₂R¹⁰;
R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR⁹, —OR¹⁰, —C(O)R¹⁰, —R¹⁰C(O)R⁹, —C(O)R⁹, —C(O)N(R^{a})(R^{b}), —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SOₘN(R^{c})(R^{d}), —SOₘCH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R⁹, —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)(R⁹), R¹⁰S—, —C(R^{a})(R^{b})NR^{a}C(R^{a})(R^{b})(R⁹), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a}), C(R^{a})(R^{b})C(O)N(R^{a})(R^{b}), —B(OH)₂, —OCH₂O— or 4,4,5,5-tetramethyl-1,3,2-dioxaborolan-2-yl; wherein said groups are optionally substituted on either the carbon or the heteroatom with one to five substituents independently selected from C₁₋₆ alkyl, halo, keto, cyano, haloalkyl, hydroxyalkyl, —OR⁹, —NO₂, —NH₂, —NHS(O)₂R⁸, —R⁹SO₂R¹², —SO₂R¹², —SO(R¹²), —SR¹², —SR⁹, —SOₘN(R^{c})(R^{d}), —SOₘN(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —C(O)(R^{a})(R^{b}), —N(R¹⁰)C(R¹⁰)(R¹¹)(R⁹), —N(R¹⁰)CO(R⁹), —NH(CH₂)₂OH, —NHC(O)OR¹⁰, —Si(CH₃)₃, heterocycyl, aryl, or heteroaryl;

### R⁸ is hydrogen or C₁₋₆ alkyl;

or R⁴ and R⁸ or can be taken together with any of the atoms to which they may be attached or are between them to form a 4-10 membered heterocyclyl ring system wherein said ring system, which may be monocyclic or bicyclic, is optionally substituted with one or two substituents independently selected from C₁₋₆ alkyl, halo, hydroxyalkyl, hydroxy, keto, —OR¹⁰, —SR¹⁰ or —N(R¹⁰)₂;
R⁹ is selected from the group consisting of hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with one, two, or three substituents independently selected from halo, alkoxy or —SO₂R¹²;

### R¹⁰ is hydrogen or C₁₋₆ alkyl

### R¹¹ is hydrogen or C₁₋₆ alkyl;

### R¹² is hydrogen or C₁₋₆ alkyl which is optionally substituted with one, two, or three substituents independently selected from halo, alkoxy, cyano, —NR¹⁰ or —SR¹⁰;

R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;
R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with one or two substituents independently selected from C₁₋₆ alkyl and halo;

### R^{c} is hydrogen or C₁₋₆ alkyl which is optionally substituted with one, two, or three substituents independently selected from halo or —OR⁹;

### R^{d} is hydrogen or C₁₋₆ alkyl which is optionally substituted with one, two, or three substituents independently selected from halo or —OR⁹;

or R^{c} and R^{d} can be taken together with the nitrogen atom to which they are attached or are between them to form a C₃₋₈ heterocyclyl ring which is optionally substituted with one or two substituents independently selected from C₁₋₆ alkyl, halo hydroxyalkyl, hydroxy, alkoxy or keto;
n is independently selected from an integer from zero to three;
each m is independently selected from an integer from zero to two;
and the pharmaceutically acceptable salts, stereoisomers and N-oxide derivatives thereof.

Nonlimiting examples of compound of formula I include:
N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide;
N¹-(1-cyanocyclopropyl)-4-fluoro-N²-{(1S)-2,2,2-trifluoro-1-[4′-(methylsulfinyl)-1,1′-biphenyl-4-yl]ethyl}-L-leucinamide;
N¹(cyanomethyl)-N²{(1S)-2,2,2-trifluoro-1-[4′-(methylsulfonyl)-1,1′-biphenyl-4-yl]ethyl}-L-leucinamide;
N²{(1S)-1-[4′-(aminosulfonyl)-1,1′-biphenyl-4-yl]-2,2,2-trifluoroethyl}-N¹(cyanomethyl)-L-leucinamide;
N¹(1-cyanocyclopropyl)-N²-{(1S)-2,2-difluoro-1-[4′-(methylsulfonyl)biphenyl-4-yl]ethyl}-4-fluoro-L-leucinamide;
   and the pharmaceutically acceptable salts thereof.

Methods of preparation for the above compounds are described in International Publication WO 03/075836, which published on Sep. 18, 2003.

Nonlimiting examples of cathepsin K inhibitors of the present invention also include:
N-(1-{[(cyanomethyl)amino]carbonyl}cyclohexyl)-4-(4-propylpiperazin-1-yl)benzamide;
N-(1-{[(cyanomethyl)amino]carbonyl}cyclohexyl)-4-[1-(2-methoxyethyl)piperidin-4-yl]benzamide;
   and the pharmaceutically acceptable salts thereof. Methods of preparation for these compounds are described in International Publication WO 99/24460, which published on May 20, 1999.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having one to ten carbon atoms unless otherwise specified. For example, C₁-C₁₀, as in “C₁-C₁₀ alkyl” is defined to include groups having 1, 2, 3, 4, 5, 6, 7, 8, 9 or 10 carbons in a linear, branched, or cyclic arrangement. For example, “C₁-C₁₀ alkyl” specifically includes methyl, ethyl, propyl, butyl, pentyl, hexyl, heptyl, octyl, nonyl, decyl, and so on.

“Alkoxy” or “alkyloxy” represents an alkyl group as defined above, unless otherwise indicated, wherein said alkyl group is attached through an oxygen bridge.

The term “cycloalkyl” or “carbocycle” shall mean cyclic rings of alkanes of three to eight total carbon atoms, unless otherwise indicated, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “cycloalkenyl” shall mean cyclic rings of 3 to 10 carbon atoms, unless otherwise specified, containing at least 1 carbon to carbon double bond (i.e., cyclopropenyl, cyclobutenyl, cyclopenentyl, cyclohexenyl, cycloheptenyl or cycloocentyl).

The term “alkynyl” refers to a hydrocarbon radical straight or branched, containing from 2 to 10 carbon atoms, unless otherwise specified, containing at least 1 carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxybenzene, benzothiazolyl, benzothienyl, quinolinyl, isoquinolinyl, oxazolyl, and tetrahydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo. The term “keto” means carbonyl (C═O). The term “alkoxy” as used herein means an alkyl portion, where alkyl is as defined above, connected to the remainder of the molecule via an oxygen atom. Examples of alkoxy include methoxy, ethoxy and the like.

The term “haloalkyl” means an alkyl radical as defined above, unless otherwise specified, that is substituted with one to five, preferably one to three halogen. Representative examples include, but are not limited to trifluoromethyl, dichloroethyl, and the like.

The term “haloalkoxy” represents a radical —OR where R is alkyl as defined above that is substituted with one to five, preferably one to three halogen. Representative examples include, but are not limited to trifluoromethyloxy, dichloroethyloxy, and the like.

The term “arylalkyl” includes an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, and chlorophenylethyl. Examples of alkylaryl include, but are not limited to, toluoyl, ethylphenyl, and propylphenyl.

The term “heteroarylalkyl” as used herein, shall refer to a system that includes a heteroaryl portion, where heteroaryl is as defined above, and contains an alkyl portion. Examples of heteroarylalkyl include, but are not limited to, thienylmethyl, thienylethyl, thienylpropyl, pyridylmethyl, pyridylethyl and imidazoylmethyl.

The term “cycloalkylalkyl” includes an alkyl portion where alkyl is as defined above and also includes a cycloalkyl portion where cycloalkyl is as defined above. Examples of cycloalkylalkyl include, but are not limited to, cyclopropylmethyl, cyclopentylmethyl, cyclohexylmethyl, cyclopropylethyl, and the like.

The term “hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, and the like.

The term “heterocycle” or “heterocyclyl” as used herein is intended to mean a 5- to 10-membered nonaromatic ring, unless otherwise specified, containing from 1 to 4 heteroatoms selected from the group consisting of O, N, S, SO, or SO₂ and includes bicyclic groups. “Heterocyclyl” therefore includes, but is not limited to the following: piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

Whenever the term “alkyl” or “aryl” or either of their prefix roots appear in a name of a substituent (e.g., aryl C₀₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The cathepsin K inhibitor compounds of the present invention can be administered in such oral dosage forms as tablets, capsules (each of which includes sustained release or timed release formulations), pills, powders, granules, elixers, tinctures, suspensions, syrups and emulsions. Likewise, they may also be administered in intravenous (bolus or infusion), intraperitoneal, topical (e.g., ocular eyedrop), subcutaneous, intramuscular or transdermal (e.g., patch) form, all using forms well known to those of ordinary skill in the pharmaceutical arts.

The dosage regimen utilizing the cathepsin K inhibitor compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the cathepsin K inhibitor compounds, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, cathepsin K inhibitor compounds may be administered in a single daily dose, or the total daily dosage may be administered in divided doses of two, three or four times daily. Furthermore, preferred cathepsin K inhibitor compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the compounds herein described can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

Exemplifying the invention is a pharmaceutical composition comprising a cathepsin K inhibitor, an anti-atherosclerotic agent and a pharmaceutically acceptable carrier. Further illustrating the invention is the use of an cathepsin K inhibitor, an anti-atherosclerotic agent and a pharmaceutically acceptable carrier for the preparation of a medicament useful in the treatment of atherosclerosis or atherosclerotic cardiovascular disease.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The cathepsin K inhibitor compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Cathepsin K inhibitor compounds may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

According to a further aspect of the present invention, it may be desirable to treat any of the aforementioned conditions with a combination of a cathepsin K inhibitor and one or more other pharmacologically active agents suitable for the treatment of atherosclerosis or atherosclerotic cardiovascular disease. The cathepsin K inhibitor and the other pharmacologically active agent(s) may be administered to a patient simultaneously, sequentially or in combination. For example, the present compound may be employed directly in combination with the other active agent(s), or it may be administered prior, concurrent or subsequent to the administration of the other active agent(s). In general, the currently available dosage forms of the known therapeutic agents for use in such combinations will be suitable.

The cathepsin K inhibitors of formula I can be used for the treatment of atherosclerosis comprising administering a therapeutically effective amount of a compound of Formula I to a patient in need of such treatment. A further aspect of this invention involves a method for preventing or reducing the risk of developing atherosclerosis, comprising administering a prophylactically effective amount of a compound of formula I to a patient in need of such treatment. Atherosclerosis is characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction, are all clinical manifestations of atherosclerosis and are therefore encompassed by the terms “atherosclerosis” and “atherosclerotic disease.”

A cathepsin K inhibitor may be administered to prevent or reduce the risk of occurrence, or recurrence where the potential exists, of a coronary heart disease event, a cerebrovascular event, and/or intermittent claudication. Coronary heart disease events are intended to include CHD (coronary heart disease), death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a cathepsin K inhibitor to a patient at risk for such an event. The patient may already have atherosclerotic disease at the time of administration, or may be at risk for developing it.

Persons to be treated with the instant therapy include those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein (HDL) cholesterol, and a family history of atherosclerotic cardiovascular disease. Published guidelines for determining those who are at risk of developing atherosclerotic disease can be found in: National Cholesterol Education Program, Second report of the Expert Panel on Detection, Evaluation, and Treatment of High Blood Cholesterol in Adults (Adult Treatment Panel II), National Institute of Health, National Heart Lung and Blood Institute, NIH Publication No. 93-3095, September 1993; abbreviated version: Expert Panel on Detection, Evaluation, and Treatment of High Blood Cholesterol in Adults, Summary of the second report of the national cholesterol education program (NCEP) Expert Panel on Detection, Evaluation, and Treatment of High Blood Cholesterol in Adults (Adult Treatment Panel II), JAMA, 1993, 269, pp. 3015-23. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The method of this invention particularly serves to prevent or slow new atherosclerotic lesion or plaque formation, and to prevent or slow progression of existing lesions or plaques, as well as to cause regression of existing lesions or plaques. Accordingly, one aspect of this invention involves a method for halting or slowing the progression of atherosclerosis, including halting or slowing atherosclerotic plaque progression, comprising administering a therapeutically effective amount of a cathepsin K inhibitor to a patient in need of such treatment. This method also includes halting or slowing progression of atherosclerotic plaques existing at the time the instant treatment is begun (i.e., “existing atherosclerotic plaques”), as well as halting or slowing formation of new atherosclerotic plaques in patients with atherosclerosis.

Another aspect of this invention involves a method for regression of atherosclerosis, including regression of atherosclerotic plaques existing at the time the instant treatment is begun, comprising administering a therapeutically effective amount of a cathepsin K inhibitor to a patient in need of such treatment. Another aspect of this invention involves a method for preventing or reducing the risk of atherosclerotic plaque rupture comprising administering a prophylactically effective amount of a cathepsin K inhibitor to a patient in need of such treatment.

In a broad embodiment, any suitable additional active agent or agents, including but not limited to anti-atherosclerotic agents, may be used in combination with the compound of formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as ZD-4522, (CRESTOR®; see U.S. Pat. No. 5,260,440; 5-lipoxygenase inhibitors; cholesterol ester transfer protein (CETP) inhibitors, for example JTT-705 and torcetrapib, also known as CP529,414; HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidinediones as well as those PPARγ agonists outside the thiazolidine dione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABCA1 gene expression; FXR and LXR ligands including both inhibitors and agonists; bisphosphonate compounds such as alendronate sodium; and cyclooxygenase-2 inhibitors such as rofecoxib and celecoxib.

Still another type of agent that can be used in combination with the compounds of this invention are cholesterol absorption inhibitors. Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A-cholesterol acyl transferase (ACAT) inhibition, inhibition of triglyceride synthesis, MTP inhibition, bile acid sequestration, and transcription modulation such as agonists or antagonists of nuclear hormones. Cholesterol absorption inhibitors are described in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 00/63703, WO/0060107, WO 00/38725, WO 00/34240, WO 00/20623, WO 97/45406, WO 97/16424, WO 97/16455, and WO 95/08532.

An exemplary cholesterol absorption inhibitor is ezetimibe, also known as SCH-58235, which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966 and shown below as

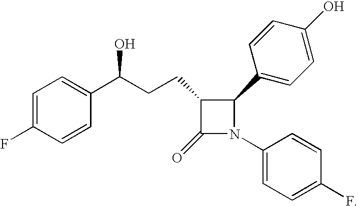

Additional exemplary hydroxy-substituted azetidinone cholesterol absorption inhibitors are specifically described in U.S. Pat. No. 5,767,115, column 39, lines 54-61 and column 40, lines 1-51, represented by the formula

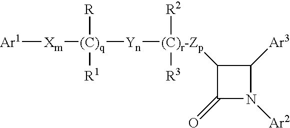

as defined in column 2, lines 20-63. These and other cholesterol absorption inhibitors can be identified according to the assay of hypolipidemic compounds using the hyperlipidemic hamster described in U.S. Pat. No. 5,767,115, column 19, lines 47-65, in which hamsters are fed a controlled cholesterol diet and dosed with test compounds for seven days. Plasma lipid analysis is conducted and data is reported as percent reduction of lipid versus control.

Therapeutically effective amounts of cholesterol absorption inhibitors include dosages of from about 0.01 mg/kg to about 30 mg/kg of body weight per day, preferably about 0.1 mg/kg to about 15 mg/kg. For an average body weight of 70 kg, the dosage level is therefore from about 0.7 mg to about 2100 mg of drug per day, e.g. 10, 20, 40, 100 or 200 mg per day, preferably given as a single daily dose or in divided doses two to six times a day, or in sustained release form. This dosage regimen may be adjusted to provide the optimal therapeutic response when the cholesterol absorption inhibitor is used in combination with a compound of the instant invention.

A therapeutically effective amount of a compound of Formula I can be used for the preparation of a medicament useful for treating or preventing any of the medical conditions described herein, in dosage amounts described herein. Additionally, the medicament may be useful for preventing or reducing the risk of developing atherosclerotic disease, halting or slowing the progression of atherosclerotic disease once it has become clinically manifest, and preventing or reducing the risk of a first or subsequent occurrence of an atherosclerotic disease event. The medicament comprised of a compound of Formula I may also be prepared with one or more additional active agents, such as those described herein.

### Assays

### Cathepsin K Assay

Serial dilutions (1/3) from 500 μM down to 0.0085 μM of test compounds are prepared in dimethyl sulfoxide (DMSO). Then 2 μL of DMSO from each dilution are added to 50 μL of assay buffer (MES, 50 mM (pH 5.5); EDTA, 2.5 mM; DTT, 2.5 mM and 10% DMSO) and 25 μL of human cathepsin K (0.4 nM) in assay buffer solution. The assay solutions are mixed for 5-10 seconds on a shaker plate and incubated for 15 minutes at room temperature. Z-Leu-Arg-AMC (8 μM) in 25 μL of assay buffer is added to the assay solutions. Hydrolysis of the coumarin leaving group (AMC) is followed by spectrofluorometry (Exλ=355 nm; Emλ=460 nm) for 10 minutes. The percent of inhibition is calculated by fitting experimental values to standard mathematical model for dose response curve.

### Effect of Cathepsin K Gene Deletion on Mouse Body Weight Increase, Percentage Body Fat and Plasma Lipids after 12 Weeks on a High Fat Diet

Cathepsin K homozygous null (KO, Saftig P et al, Proc Natl Acad Sci USA. 1998; 95:13453-8) and wild-type littermate controls (WT, mixed 129/C57BL/6 background) were fed a high fat diet (HFD, 35% fat, 35% carbohydrate by weight, Bio-Serv (F3282), n=10-12/group) for 12 weeks starting at an age of age approximately 8 weeks. The animals were weighed weekly and food consumption was determined. No difference in food consumption between groups was detected. The percentage of body fat for each animal was determined using dual energy X-ray absorptiometry after 12 weeks on the HFD. At termination, blood was taken for determination of plasma cholesterol triglycerides and leptin.

| | Male WT | Male KO | Female WT | Female KO |
|---|---|---|---|---|
| Body weight gain | 14.9 ± 2.0^{a} | 12.9 ± 1.8 | 13.4 ± 2.3 | 6.8 ± 1.6** |
| after 12 weeks on | | | | |
| HFD | | | | |
| % body fat after | 32.8 ± 2.7 | 32.5 ± 1.0 | 33.7 ± 3.8 | 22.1 ± 2.8* |
| 12 weeks on HFD | | | | |
| Plasma cholesterol | 5.19 ± 0.24 | 3.65 ± 0.27** | 3.73 ± 0.27 | 3.14 ± 0.12* |
| (mM) | | | | |
| Plasma | 1.76 ± 0.11 | 1.42 ± 0.07* | 1.64 ± 0.17 | 1.59 ± 0.08 |
| triglycerides (mM) | | | | |
| Plasma leptin | 83.75 ± 14.46 | 37.25 ± 5.30* | 32.24 ± 8.73 | 13.68 ± 5.91* |
| (ng/mL) | | | | |

^{a}±SEM.
*p < 0.05,
**p < 0.01, vs WT

### Apolipoprotein E-Deficient Mice

Apolipoprotein E-deficient mouse models are described in Plump A S, Smith J D, Hayek T, Aalto-Setala K, Walsh A, Verstuyft J G, Rubin E M, Breslow J L. Severe hypercholesterolemia and atherosclerosis in apolipoprotein E-deficient mice created by homologous recombination in ES cells. Cell. (1992) 71: 343-53; and Williams H, Johnson J L, Carson K G, Jackson C L. Characteristics of intact and ruptured atherosclerotic plaques in brachiocephalic arteries of apolipoprotein F knockout mice. Arterioscler Thromb Vasc Biol. (2002) 22:788-92.

### Low Density Lipoprotein Receptor Knockout Mice

Low density lipoprotein receptor knockout mouse models are described in Ishibashi, S., Brown, M S, Goldstein, J L, Gerard, R D, Hammer, R E and Herz, J. Hypercholesterolemia in low density lipoprotein receptor knockout mice and its reversal by adenovirus-mediated gene delivery. J. Clin. Invest. (1993) 92: 883-893.

### Watanabe-Heritable Hyperlipidemic Rabbit

The Watanabe-heritable hyperlipidemic rabbit is described in Watanabe Y. Serial inbreeding of rabbits with hereditary hyperlipidemia (WHHL-rabbit). Atherosclerosis. (1980) 36: 261-8.

What is claimed:

1. A method for treating atherosclerosis or atherosclerotic cardiovascular disease comprising administering a therapeutically effective amount of a cathepsin K inhibitor.

2. The method of claim 1 wherein the cathepsin K inhibitor is represented by formula I: 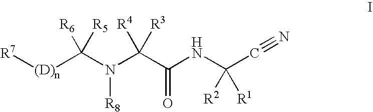
wherein R¹ is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
R² is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
or R¹ and R² can be taken together with the carbon atom to which they are attached to form a C₃₋₈ cycloalkyl or heterocyclyl ring wherein said ring system is optionally substituted with one or two substituents independently selected from C₁₋₆ alkyl, hydroxyalkyl, haloalkyl, or halo;
R³ is hydrogen, C₁₋₆ alkyl or C₂₋₆ alkenyl wherein said alkyl and alkenyl groups are optionally substituted with C₃₋₆ cycloalkyl or one to six halo;
R⁴ is hydrogen, C₁₋₆ alkyl or C₂₋₆ alkenyl wherein said alkyl and alkenyl groups are optionally substituted with C₃₋₆ cycloalkyl or one to six halo;
or R³ and R⁴ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted with one or two substitutents independently selected from C₁₋₆ alkyl, halo, hydroxyalkyl, hydroxy, alkoxy or keto;
R⁵ is selected from hydrogen or C₁₋₆ alkyl substituted with 1-6 halo;
R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with one, two, or three substituents independently selected from halo, C₁₋₆ alkyl, C₁₋₆ haloalkyl, C₃₋₆ cycloalkyl, haloalkoxy, —SR⁹, —SR¹², —SOR⁹, —SOR¹², —SO₂R⁹, —SO₂R¹², —SO₂CH(R¹²)(R¹¹), —OR¹², —N(R¹⁰)(R¹¹), cyano, or aryl which is optionally substituted with —SO₂R¹²;
each D is independently C₁₋₃ alkyl, C₂₋₃ alkenyl, C₂₋₃ alkynyl, aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from C₁₋₆ alkyl, haloalkyl, halo, keto, alkoxy, —SR⁹, —SR¹², —OR⁹, —OR¹², N(R¹²)₂, —SO₂R⁹, or —SO₂R¹⁰;
R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR⁹, —OR¹⁰, —C(O)R¹⁰, —R¹⁰C(O)R⁹, —C(O)R⁹, —C(O)N(R^{a})(R^{b}), —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², SO(R¹²), —SO₂R⁹, —SOₘN(R^{c})(R^{d}), —SOₘCH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R⁹, —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)(R⁹), R¹⁰S—, —C(R^{a})(R^{b})NR^{a}C(R^{a})(R^{b})(R⁹), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a}), C(R^{a})(R^{b})C(O)N(R^{a})(R^{b}), —B(OH)₂, —OCH₂O— or 4,4,5,5-tetramethyl-1,3,2-dioxaborolan-2-yl; wherein said groups are optionally substituted on either the carbon or the heteroatom with one to five substituents independently selected from C₁₋₆ alkyl, halo, keto, cyano, haloalkyl, hydroxyalkyl, —OR⁹, —NO₂, —NH₂, —NHS(O)₂R⁸, —R⁹SO₂R¹², —SO₂R¹², —SO(R¹²), —SR¹², —SR⁹, —SOₘN(R^{c})(R^{d}), —SOₘN(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —C(O)(R^{a})(R^{b}), —N(R¹⁰)C(R¹⁰)(R¹¹)(R⁹), —N(R¹⁰)CO(R⁹), —NH(CH₂)₂OH, —NHC(O)OR¹⁰, —Si(CH₃)₃, heterocycyl, aryl, or heteroaryl;
R⁸ is hydrogen or C₁₋₆ alkyl;
or R⁴ and R⁸ or can be taken together with any of the atoms to which they may be attached or are between them to form a 4-10 membered heterocyclyl ring system wherein said ring system, which may be monocyclic or bicyclic, is optionally substituted with one or two substituents independently selected from C₁₋₆ alkyl, halo, hydroxyalkyl, hydroxy, keto, —OR¹⁰, —SR¹⁰ or —N(R¹⁰)₂;
R⁹ is selected from the group consisting of hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with one, two, or three substituents independently selected from halo, alkoxy or —SO₂R¹²;
R¹⁰ is hydrogen or C₁₋₆ alkyl;
R¹¹ is hydrogen or C₁₋₆ alkyl;
R¹² is hydrogen or C₁₋₆ alkyl which is optionally substituted with one, two, or three substituents independently selected from halo, alkoxy, cyano, —NR¹⁰ or —SR¹⁰;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;
R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with one or two substituents independently selected from C₁₋₆ alkyl and halo;
R^{c} is hydrogen or C₁₋₆ alkyl which is optionally substituted with one, two, or three substituents independently selected from halo or —OR⁹;
R^{d} is hydrogen or C₁₋₆ alkyl which is optionally substituted with one, two, or three substituents independently selected from halo or —OR⁹;
or R^{c} and R^{d} can be taken together with the nitrogen atom to which they are attached or are between them to form a C₃₋₈ heterocyclyl ring which is optionally substituted with one or two substituents independently selected from C₁₋₆ alkyl, halo hydroxyalkyl, hydroxy, alkoxy or keto;
n is independently selected from an integer from zero to three;
each m is independently selected from an integer from zero to two;
and the pharmaceutically acceptable salts, stereoisomers and N-oxide derivatives thereof.

3. The method of claim 2 wherein the cathepsin K inhibitor is
N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide;
N¹-(1-cyanocyclopropyl)-4-fluoro-N²-{(1S)-2,2,2-trifluoro-1-[4′-(methylsulfinyl)-1,1′-biphenyl-4-yl]ethyl}-L-leucinamide;
N¹(cyanomethyl)-N²{(1S)-2,2,2-trifluoro-1-[4′-(methylsulfonyl)-1,1′-biphenyl-4-yl]ethyl}-L-leucinamide;
N²{(1S)-1-[4′-(aminosulfonyl)-1,1′-biphenyl-4-yl]-2,2,2-trifluoroethyl}-N¹(cyanomethyl)-L-leucinamide;
N¹(1-cyanocyclopropyl)-N²-{(1S)-2,2-difluoro-1-[4′-(methylsulfonyl)biphenyl-4-yl]ethyl}-4-fluoro-L-leucinamide;
or a pharmaceutically acceptable salt thereof.

4. The method of claim 1 wherein the cathepsin K inhibitor is
N-(1-{[(cyanomethyl)amino]carbonyl}cyclohexyl)-4-(4-propylpiperazin-1-yl)benzamide;
N-(1-{[(cyanomethyl)amino]carbonyl}cyclohexyl)-4-[1-(2-methoxyethyl)piperidin-4-yl]benzamide;
or a pharmaceutically acceptable salt thereof.

5. The method of claim 1 for halting or slowing atherosclerotic plaque progression.

6. The method of claim 1 for effecting regression of atherosclerotic plaque.

7. The method of claim 1 for preventing or reducing the risk of atherosclerotic plaque rupture in a patient having atherosclerotic plaque.

8. The method of claim 1 wherein the atherosclerotic cardiovascular disease is restenosis following revascularization procedures, coronary heart disease, cerebrovascular disease or peripheral vessel disease.

9. The method of claim 2 further comprising administering to the patient a compound selected from the group consisting of an HMG-CoA reductase inhibitor, cholesterol absorption inhibitor, CETP inhibitor, PPARγ agonist, PPARα agonist, PPAR dual α/γ agonist, and combinations thereof.

10. A pharmaceutical composition comprising a cathepsin K inhibitor and an anti-atherosclerotic agent.

11. The pharmaceutical composition of claim 9 wherein the anti-atherosclerotic agent is selected from the group consisting of an HMG-CoA reductase inhibitor, cholesterol absorption inhibitor, CETP inhibitor, PPARγ agonist, PPARα agonist, PPAR dual α/γ agonist, and combinations thereof.

12. Use of a cathepsin K inhibitor in the manufacture of a medicament for treating atherosclerosis, or atherosclerotic cardiovascular disease.

13. Use according to claim 12, wherein said cathepsin K inhibitor is as defined in claim 2, 3 or 4.

14. A cathepsin K inhibitor for use in treating atherosclerosis, or atherosclerotic cardiovascular disease.

15. A cathepsin K inhibitor according to claim 14, as defined in claim 2, 3 or 4.
